(19) United States
(12) Patent Application Publication
(10) Pub. No.: US 2025/0025543 A1
(43) Pub. Date: Jan. 23, 2025

(54) DRUG FOR TREATING AND/OR PREVENTING CANCER AND USE THEREOF

(71) Applicant: YUANBEN (ZHUHAI HENGQIN) BIOTECHNOLOGY CO., LTD., Zhuhai, Guangdong (CN)

(72) Inventor: Jiong CAI, Taizhou, Jiangsu (CN)

(21) Appl. No.: 18/706,821

(22) PCT Filed: Aug. 25, 2022
(86) PCT No.: PCT/CN2022/114957

§ 371 Date: May 2, 2024

(30) Foreign Application Priority Data
Nov. 4, 2021 (CN) 202111302270.4

(51) Int. Cl. A61K 39/00; A61K 31/282; A61K 39/395; A61P 37/04

(52) U.S. Cl. CPC A61K 39/00117; A61K 31/282; A61K 39/3955; A61P 37/04

(57) ABSTRACT

The present invention provides a drug for treating and/or preventing cancer and a use thereof. The drug in the present invention comprises a recombinant MBP-MUC1-N fusion protein vaccine, a PD-1 antibody and/or oxaliplatin, and the drug can enhance an MUC1-specific anti-tumor immune response.

The present application claims priority to the prior application with the patent application No. 2021113022704 entitled “DRUG FOR TREATING AND/OR PREVENTING CANCER AND USE THEREOF” filed to the China National Intellectual Property Administration on Nov. 4, 2021, which is incorporated herein by reference in its entirety.

### TECHNICAL FIELD

The present disclosure relates to the technical field of immunotherapeutic medicaments, in particular to a medicament for treating and/or preventing cancer and use thereof.

### BACKGROUND

Cancer vaccines are active immunotherapy, which specifically kill cancer cells by targeting tumor antigens. Cancer vaccines mainly include DNA vaccines, DC vaccines, oncolytic virus vaccines or recombinant virus vector vaccines, and vaccines based on peptides or proteins. To date, most studies on cancer vaccines are in phase I to phase II clinical trials, with over 10 products entering phase III clinical trials, and two therapeutic cancer vaccines are on the market: one was the Provenge vaccine approved by the U.S. FDA for the treatment of prostate cancer in 2010, and the other was the T-VEC vaccine approved by the U.S. FDA for the treatment of advanced colon cancer in 2015. The two cancer vaccines prolonged the survival of patients by 4 months and 4.4 months, respectively, in phase III clinical trials, and could only generate modest clinical benefit. The Provenge vaccine was also controversial in use because of the inability to prolong the disease progression time in patients and the high cost. In addition, the phase III clinical trials for the PROSTVAC recombinant virus vector vaccine and for the MAGE-A3 fusion protein vaccine were terminated due to the lack of clinical benefits to patients in the phase III clinical trials. Cancer vaccines have long been conceived to enhance tumor-specific immune responses, particularly T cell responses, by active immunization and as a key tool for effective cancer immunotherapy. However, due to the existence of immune tolerance microenvironments in vivo, cancer vaccines can only generate modest clinical benefits. Therefore, it is particularly important to overcome immune tolerance microenvironments and improve clinical benefits of vaccines.

In the aspect of cancer vaccine monotherapy, only modest clinical benefits can be generated, the immune tolerance microenvironments cannot be overcome. PD-1 antibodies can make up for the defects of the cancer vaccines and overcome the immune tolerance microenvironments, thereby exciting tumor antigen-specific immune responses, and specifically killing tumor cells. In the aspect of PD-1 monotherapy, the objective response rate thereof in most cancers is only about 20%-30%, and this monotherapy has a significant therapeutic effect on tumors with tumor infiltration immune responses before, but has a poor effect on non-immunogenic tumors, lacking specificity for killing tumors, and easily causing autoimmunity. Cancer vaccines can just make up for the deficiency of the PD-1 antibodies, the cancer vaccines can generate immune responses at tumor sites, and the vaccine-mediated tumor cell death leads to the release of more cascade antigens. The combined application of the cancer vaccines and the PD-1 antibodies can complement each other, thereby improving the tumor-killing activity. Based on the theoretical basis described above, the combination of the cancer vaccine with the PD-1 antibody has become one of the hot spots of current studies. To date, the combination of the cancer vaccine with the PD-1 antibody has been in an early stage of research, in which T-VEC in combination with pembrolizumab (a PD-1 antibody) was in phase I clinical trials in patients with advanced colon cancer and advanced head and neck squamous cell carcinoma, the objective response rate of the combination therapy was significantly improved compared to the monotherapy, and especially in patients with advanced colon cancer, the objective response rate of the combination therapy was 2-3 times that of the monotherapy. Moreover, the combination therapy has controlled safety. In addition, phase I clinical trials of Nivolumab (a PD-1 antibody) in combination with a peptide vaccine (MART-1/NY-ESO-1/gp100 in combination with Montanide) in patients with advanced colon cancer showed that the relapse-free survival rate of the combination therapy was twice that of the monotherapy of vaccines (47.1 months vs. 12-21 months). However, there were also cases of failure of the cancer vaccines in combination with the PD-1 antibodies, in which the PD-1 antibodies in combination with the peptide vaccines failed to improve the clinical efficacy of PD-1 monotherapy in phase I clinical trials for refractory and treatment-naive colon cancer. Therefore, the combination of cancer vaccines with PD-1 antibodies needs an infusion of fresh blood urgently.

### SUMMARY

The object of the present disclosure is to provide a medicament for treating and/or preventing cancer and use thereof. The medicament of the present disclosure combines a recombinant MBP-MUC1-N fusion protein vaccine with an anti-PD-1 antibody and/or oxaliplatin, which is expected to eliminate the immune tolerance state of immune cells in a tumor microenvironment, especially T cells, enhances an MUC1-specific anti-tumor immune response, and is expected to completely eliminate tumors, bringing good news for cancer patients.

The present disclosure provides a medicament for treating and/or preventing cancer, which comprises a recombinant MBP-MUC1-N fusion protein vaccine and an anti-PD-1 antibody.

Preferably, the recombinant MBP-MUC1-N fusion protein vaccine comprises maltose-binding protein MBP gene and human mucin MUC1-N gene in tandem.

More preferably, the nucleotide sequence of the MUC1-N gene is set forth in SEQ ID NO. 1, and preferably, the MBP gene is set forth in SEQ ID NO. 2.

Preferably, the cancer comprises all cancers expressing MUC1, including adenocarcinomas expressing MUC1 or hematological tumors expressing MUC1.

The present disclosure further provides use of a recombinant MBP-MUC1-N fusion protein vaccine in combination with an anti-PD-1 antibody in the manufacture of a medicament for preventing and/or treating cancer.

Preferably, the recombinant MBP-MUC1-N fusion protein vaccine comprises maltose-binding protein MBP gene and human mucin MUC1-N gene in tandem.

More preferably, the nucleotide sequence of the MUC1-N gene is set forth in SEQ ID NO. 1, and preferably, the MBP gene is set forth in SEQ ID NO. 2.

Preferably, the cancer comprises all cancers expressing MUC1, including adenocarcinomas expressing MUC1 or hematological tumors expressing MUC1.

The present disclosure further provides a medicament for treating and/or preventing cancer, which characterized in that the medicament comprises a recombinant MBP-MUC1-N fusion protein vaccine and oxaliplatin.

Preferably, the recombinant MBP-MUC1-N fusion protein vaccine comprises maltose-binding protein MBP gene and human mucin MUC1-N gene in tandem.

More preferably, the nucleotide sequence of the MUC1-N gene is set forth in SEQ ID NO. 1, and preferably, the MBP gene is set forth in SEQ ID NO. 2.

Preferably, the cancer comprises all cancers expressing MUC1, including adenocarcinomas expressing MUC1 or hematological tumors expressing MUC1, preferably colorectal cancer or colon cancer.

The present disclosure further provides use of a recombinant MBP-MUC1-N fusion protein vaccine in combination with oxaliplatin in the manufacture of a medicament for preventing and/or treating cancer.

Preferably, the recombinant MBP-MUC1-N fusion protein vaccine comprises maltose-binding protein MBP gene and human mucin MUC1-N gene in tandem.

More preferably, the nucleotide sequence of the MUC1-N gene is set forth in SEQ ID NO. 1, and preferably, the MBP gene is set forth in SEQ ID NO. 2.

Preferably, the cancer comprises all cancers expressing MUC1, including adenocarcinomas expressing MUC1 or hematological tumors expressing MUC1, preferably colorectal cancer or colon cancer.

The present disclosure further provides use of a recombinant MBP-MUC1-N fusion protein vaccine in combination with oxaliplatin in the manufacture of a medicament for delaying a cancer patient.

Beneficial Effects of Present Disclosure: The present disclosure provides a medicament for treating and/or preventing cancer and use thereof.

The medicament of the present disclosure combines a recombinant MBP-MUC1-N fusion protein vaccine with an anti-PD-1 antibody, which can remarkably enhance the anti-tumor effect of the recombinant MBP-MUC1-N fusion protein vaccine. The medicament of the present disclosure can effectively inhibit the growth of colon cancer expressing MUC1. Test results show that compared to a monotherapy of an anti-PD-1 antibody or a recombinant MUC1 fusion protein vaccine, the combination of the recombinant MBP-MUC1-N fusion protein vaccine with the anti-PD-1 antibody can significantly improve the tumor inhibition rate, which is more than, and significantly improve the tumor cure rate, which is between, so that the combination of the recombinant MBP-MUC1-N fusion protein vaccine with the anti-PD-1 antibody is expected to overcome the immune tolerance state in the tumor microenvironment, enhances the MUC1-specific anti-tumor immune response, and is expected to completely eliminate tumors, bringing good news for cancer patients.

In addition, the medicament of the present disclosure combines the recombinant MBP-MUC1-N fusion protein vaccine with oxaliplatin, which can remarkably enhance the anti-tumor effect of the recombinant MBP-MUC1-N fusion protein vaccine. The medicament of the present disclosure can effectively inhibit the growth of colon cancer expressing MUC1. Test results show that compared to the monotherapy of oxaliplatin or the recombinant MUC1 fusion protein vaccine, the combination of the recombinant MBP-MUC1-N fusion protein vaccine with oxaliplatin can significantly improve the tumor inhibition rate, which is more than, and significantly improve the tumor cure rate, which is between, so that the combination of the recombinant MBP-MUC1-N fusion protein vaccine with oxaliplatin is expected to overcome the immune tolerance state in the tumor microenvironment, enhances the MUC1-specific anti-tumor immune response, and is expected to completely eliminate tumors, bringing good news for cancer patients.

### BRIEF DESCRIPTION OF THE DRAWINGS

FIG. 1 shows the result of the inhibitory effect of the recombinant MBP-MUC1-N fusion protein vaccine provided by the present disclosure in combination with the anti-PD-1 antibody on MC38 colon cancer;

FIG. 2 shows the result of the inhibitory effect of the recombinant MBP-MUC1-N fusion protein vaccine provided by the present disclosure in combination with oxaliplatin on MC38 colon cancer;

FIG. 3 shows the results of the effect of the recombinant MBP-MUC1-N fusion protein vaccine provided by the present disclosure in combination with oxaliplatin on the life of cancer mice;

FIG. 4 shows the results of the inhibitory effect of the recombinant MBP-MUC1-N fusion protein vaccine provided by the present disclosure on MC38 colon cancer;

FIG. 5 shows effects of different buffers on the efficacy of vaccines in treating MC38 colon cancer.

### DETAILED DESCRIPTION

The present disclosure provides a medicament for treating and/or preventing cancer, which comprises a recombinant MBP-MUC1-N fusion protein vaccine and an anti-PD-1 antibody and/or oxaliplatin. In the present disclosure, the source of the anti-PD-1 antibody and/or oxaliplatin is not particularly limited, and a conventional commercially available anti-PD-1 antibody and/or oxaliplatin may be used.

### 1. Provided is Preparation of a Recombinant MBP-MUC1-N Fusion Protein Vaccine, Wherein the Nucleotide Sequence of an MUC1-N Protein is Set Forth in SEQ ID NO. 1:

ggtgttacttctgctcctgatactcgtcctgctcctggttctactgcac
cgccagcacatggcgtgacgtctgcgccagatacccgtccggcaccggg
ttccaccgccccaccggcacacggcgtaacctccgcgccagacacccgt
ccagcgccaggttctaccgctccgcctgctcatggtgttacctctgccc
cggacactcgtccggctccaggttctactgccccgccagctcatggcgt
cacttccgccccggatacccgtcctgccccgggctctactgcgcctccg
gctcacggcgttacctctgcaccggatactcgtccggctccgggctcta
ccgcaccacctgctcatggcgtaacgagcgctcctgatacccgtccggc
tccgggttccactgcacctccggcccac

The nucleotide sequence of an MBP protein is set forth in SEQ ID NO. 2:

aaaatcgaagaaggcaaactggtgatctggatcaacggtgataagggtt
ataacggtctggcggaagtaggcaagaaattcgaaaaagacaccggtat
caaagttaccgttgaacatccagacaaactggaagaaaaattccctcag
gtggcggctaccggcgacggccctgatatcattttctgggcacatgatc
gttttggcggttacgcgcagtctggcctgctggcagaaatcacgccgga
taaggcgttccaggacaaactgtacccttttacctgggacgcggtgcgt
tacaacggcaaactgatcgcttacccgatcgcagtggaagctctgtccc
tgatctacaataaggacctgctgccgaacccgcctaaaacgtgggaaga
aatcccggccctggacaaagaactgaaagcaaaaggtaagagcgctctg
atgttcaatctgcaggaaccgtacttcacttggccgctgatcgcagctg
acggcggttatgcgtttaaatacgaaaacggtaaatatgacattaagga
cgtcggcgttgataacgccggcgccaaagcgggcctgacctttctggtc
gacctgatcaaaaacaaacacatgaacgctgacaccgattattctattg
cggaggcggcttttaacaagggcgagaccgcaatgaccatcaacggtcc
gtgggcttggtctaacatcgacacctccaaagtaaattacggtgttacc
gtcctgccgaccttcaaaggtcaaccgagcaaaccgttcgtgggcgtgc
tgtccgcaggtatcaacgctgcctccccaaacaaagagctggccaaaga
gttcctggaaaactatctgctgaccgacgaaggcctggaagctgttaat
aaagacaaaccgctgggtgctgttgcactgaaatcctatgaagaagaac
tggtcaaagatccgcgtattgccgccactatggagaacgcgcagaaagg
tgaaatcatgccgaacatcccgcaaatgtccgctttttggtacgcggtg
cgtaccgctgtaattaacgcggcgtccggtcgtcagactgtcgatgaag
cgctgaaagatgctcagactaactctagctctaacaataacaataataa
caacaacaacaatctgggtattgaaggtcgcatctct

### Synthesis of Optimized Gene Sequence of MUC1-N Fused to MBP

To achieve sequential tandem expression of MBP and Muc1-N, a protein with a fusion protein sequence (SEQ ID NO. 3)

KIEEGKLVIWINGDKGYNGLAEVGKKFEKDTGIKVTVEHPDKLEEKFPQ
VAATGDGPDIIFWAHDRFGGYAQSGLLAEITPDKAFQDKLYPFTWDAVR
YNGKLIAYPIAVEALSLIYNKDLLPNPPKTWEEIPALDKELKAKGKSAL
MFNLQEPYFTWPLIAADGGYAFKYENGKYDIKDVGVDNAGAKAGLTFLV
DLIKNKHMNADTDYSIAEAAFNKGETAMTINGPWAWSNIDTSKVNYGVT
VLPTFKGQPSKPFVGVLSAGINAASPNKELAKEFLENYLLTDEGLEAVN
KDKPLGAVALKSYEEELVKDPRIAATMENAQKGEIMPNIPQMSAFWYAV
RTAVINAASGRQTVDEALKDAQTNSSSNNNNNNNNNNLGIEGRISGVTS
APDTRPAPGSTAPPAHGVTSAPDTRPAPGSTAPPAHGVTSAPDTRPAPG
STAPPAHGVTSAPDTRPAPGSTAPPAHGVTSAPDTRPAPGSTAPPAHGV
TSAPDTRPAPGSTAPPAHGVTSAPDTRPAPGSTAPPAH

was obtained. Tandem synthesis was performed based on the gene sequence of the MBP and Muc1-N fusion protein. For this purpose, oligonucleotide sequences 1a_1, 1a_2, 1a_3, 1a_4, 1a_5, 1a_6, 1a_7, 1a_8, 1a_9, 1a_10, 1a_11, 1a_12, 1a_13, 1a_14, 1a_15, 1a_16, 1a_17, 1a_18, 1a_19, 1a_20, 1a_21, 1a_22, 1a_23, 1a_24, 1a_25, 1a_26, 1a_27, 1a_28, 1a_29, and 1a_30 were synthesized first, then sequences 1b_1, 1b_2, 1b_3, and 1b_4 were synthesized, and gene amplification was performed using 1-seq2 and 1-R sequences to obtain the optimized gene sequence of MUC1-N fused to MBP.

| | | Number |
|---|---|---|
| No. | Sequence | of bases |
| 1-seq2 | tacttcacttggccgctgat | 20 |
| 1-R | ggcctctgcagtcgacgggcccggggaagacttggacgcgtatccggtgcagaagtaacgccgtgtgcc | 116 |
| | ggcggtgcagtggaacccggcgctggacgagtatccggcgcagacgt | |
| 1a_1 | aacgacggccagagaattcgagctcggtaccggatccctcctcgctgcccagccggcgatggcc | 64 |
| 1a_2 | cccttatcaccgttgatccagatcaccagtttgccttcttcgattttatccatggccatcgccggctg | 68 |
| 1a_3 | caacggtgataagggttataacggtctggcggaagtaggcaagaaattcgaaaaagacaccggtatca | 68 |
| 1a_4 | acctgagggaatttttcttccagtttgtctggatgttcaacggtaactttgataccggtgtctt | 64 |
| 1a_5 | aaaattccctcaggtggcggctaccggcgacggccctgatatcattttctgggcacatgatcgttttg | 68 |
| 1a_6 | aacgccttatccggcgtgatttctgccagcaggccagactgcgcgtaaccgccaaaacgatcatgtg | 67 |
| 1a_7 | gccggataaggcgttccaggacaaactgtacccttttacctgggacgcggtgcgttacaacggcaa | 66 |
| 1a_8 | cttattgtagatcagggacagagcttccactgcgatcgggtaagcgatcagtttgccgttgtaacgc | 67 |
| 1a_9 | ctgatctacaataaggacctgctgccgaacccgcctaaaacgtgggaagaaatcccggccctggacaaa | 69 |
| 1a_10 | cggttcctgcagattgaacatcagagcgctcttaccttttgctttcagttctttgtccagggccgg | 66 |
| 1a_11 | aatctgcaggaaccgtacttcacttggccgctgatcgcagctgacggcggttatgcgtttaaatacg | 67 |
| 1a_12 | tttggcgccggcgttatcaacgccgacgtccttaatgtcatatttaccgttttcgtatttaaacgcat | 68 |
| 1a_13 | aacgccggcgccaaagcgggcctgacctttctggtcgacctgatcaaaaacaaacacatgaacgct | 66 |
| 1a_14 | gcggtctcgcccttgttaaaagccgcctccgcaatagaataatcggtgtcagcgttcatgtgttt | 65 |
| 1a_15 | caagggcgagaccgcaatgaccatcaacggtccgtgggcttggtctaacatcgacacctccaa | 63 |
| 1a_16 | ttgctcggttgacctttgaaggtcggcaggacggtaacaccgtaatttactttggaggtgtcgatg | 66 |
| 1a_17 | aggtcaaccgagcaaaccgttcgtgggcgtgctgtccgcaggtatcaacgctgcctccccaaacaaa | 67 |
| 1a_18 | ccttcgtcggtcagcagatagttttccaggaactctttggccagctctttgtttggggaggc | 62 |
| 1a_19 | gctgaccgacgaaggcctggaagctgttaataaagacaaaccgctgggtgctgttgcactgaaa | 64 |
| 1a_20 | ctccatagtggcggcaatacgcggatctttgaccagttcttcttcataggatttcagtgcaacagc | 66 |
| 1a_21 | gccgccactatggagaacgcgcagaaaggtgaaatcatgccgaacatcccgcaaatgtccgc | 62 |
| 1a_22 | ctgacgaccggacgccgcgttaattacagcggtacgcaccgcgtaccaaaaagcggacatttgcggg | 67 |
| 1a_23 | gcgtccggtcgtcagactgtcgatgaagcgctgaaagatgctcagactaactctagctctaaca | 64 |
| 1a_24 | gagatgcgaccttcaatacccagattgttgttgttgttattattgttattgttagagctagagt | 64 |
| 1a_25 | tgaaggtcgcatctctggcgttactagcgcaccggatacccgtccggcaccgggctctaccgct | 64 |
| 1a_26 | cggagcggaagtaacaccgtgagcaggcggagcggtagagcccgg | 45 |
| 1a_27 | tgttacttccgctccggacacccgtccagcgccaggttccaccgcaccgccggcacacggcgttacctccg | 86 |
| | ctccagatactcgcc | |
| 1a_28 | agtatccggcgcagacgtgacgccgtgcgctggcggtgcagtagaacccggtgccgggcgagtatctgg | 71 |
| | ag | |
| 1a_29 | tctgcgccggatactcgtccagcgccgggttccactgcaccgccggcacacggcgttacttctgcaccgga | 89 |
| | tacgcgtccaagtcttcc | |
| 1a_30 | ggcctctgcagtcgacgggcccggggaagacttggacgc | 39 |
| 1b_1 | ccaatggtctcagtccggctccaggttctactgccccgccggcacatggcgttacctctg | 60 |
| 1b_2 | cgtgcgccggaggagcggtgctgcctggtgcagggcgagtgtccggggcagaggtaacgccat | 63 |
| 1b_3 | ctcctccggcgcacggtgtcacttctgctccagacaccc | 39 |
| 1b_4 | ggccgcaagcttgtcgacggagctcgaattctcagtgcgccggggggggtgctgcccggagccggac | 84 |
| | gggtgtctggagcag | |

### 2. Recombinant Expression and Purification of Proteins

A NcoI restriction site was added to a 5′ PCR primer of the fusion gene, and an EcoI restriction site was added to a 3′ PCR primer. The amplified gene was subjected to double digestion, and then inserted into a pET26b(+) Escherichia coli expression vector subjected to the same double digestion. The bacteria were screened in resistant bacterial culture plates and subjected to monoclonal selection. The bacteria were cultured using a kanamycin-resistant medium, and the expression of the operon was induced by IPTG. The non-optimized sequence and the optimized sequence were subjected to experiments. The obtained final whole bacterial liquid was pretreated with a buffer containing SDS at 95° C. and analyzed by 5%-12% polyacrylamide gel electrophoresis. The results show that: the expression level of the MBP-Muc1-N sequence before optimization was only 2% of the total protein, and the expression level of the 1MBP-Muc1-N sequence after optimization accounted for 51% of the total protein, which was increased by 25.5 times. After purification by an affinity column, the protein expressed by the non-optimized gene must be subjected to 10-fold concentration before loading for observation, the yield of the purified protein after concentration was 0.8 mg of 100 mL culture, and the yield of the optimized MBP-Muc1-N sequence was 9.6 mg, which was increased by 12 times. The medicament for treating and/or preventing cancer and use thereof described herein will be further described in detail below with reference to specific examples, and the technical solutions of the present disclosure include, but are not limited to, the following examples.

### Example 1. Treatment of MC38 Colon Cancer in Combination with PD-1 Antibody

### 1. Materials

Experimental reagents: the recombinant MBP-MUC1-N fusion protein prepared by using the method of the present application, and the vaccine was prepared by adopting a conventional vaccine 5 preparation method; the anti-PD-1 antibody was purchased from Shanghai Junshi Biosciences Co., Ltd.; MC38 colon cancer cells were purchased from National Experimental Cell Resource Center; the normal saline for injection was purchased from Beijing Tiantan Biological Products Co., Ltd. Experimental animals: C57 BL/6J mice were purchased from Beijing Huafukang Biotechnology Co., Ltd.

### 2. Methods

### 2.1 Immunization of Mice

Mice were weighed and randomly divided into groups of 6. MC38 colon cancer cells were diluted with an IMDM basal medium and inoculated in the right axilla of the C57BL/J mice according to 5×10⁵ cells/mouse, with a total amount of 100 μL/mouse, for modeling. After the tumor diameter reached 5 mm, the mice were divided into 4 groups, namely a normal saline control group (NS group), an immune checkpoint inhibitor anti-PD-1 antibody injection group (PD-1 group), a recombinant MBP-MUC1-N fusion protein vaccine group (vaccine group), and a recombinant MBP-MUC1-N fusion protein vaccine formulation in combination with immune checkpoint inhibitor anti-PD1 antibody group (combination group). On day 3, day 7, day 10, day 14, and day 17 of tumor bearing, the recombinant MBP-MUC1-N fusion protein vaccine formulation was intramuscularly injected at a dose of 100 μg/mouse (diluted with NS, with a total amount of 200 μL) for immunization, and the immune checkpoint inhibitor anti-PD-1 antibody was intraperitoneally injected at a dose of 250 μg/mouse (diluted with NS, with a total amount of 500 tL/mouse). 2.2 Inhibitory effect of recombinant MBP-MUC1-N fusion protein vaccine in combination with anti-PD-1 antibody on MC38 colon cancer

The tumor graft inhibition rate and the tumor inhibition rate of the subcutaneous colon cancer in each group were calculated. The formula is as follows: [tumor inhibition rate=(mean tumor weight in control group −mean tumor weight in experimental group)/mean tumor weight in control group×100%]. [tumor cure rate=(number of mice without tumor/total number of mice)×100%].

### 3. Results

TABLE 1
Tumor weights (g), tumor inhibition rates
(%) and tumor cure rates in mice

| | Tumor | Tumor | Tumor |
|---|---|---|---|
| | weight | inhibition rate | cure rate |
| NS group | 2.64 ± 0.64 | 0 | 0 |
| PD-1 group | 1.33 ± 0.63 | 50 | 17 |
| Vaccine group | 1.96 ± 0.80 | 26 | 0 |
| Combination group | 0.34 ± 0.23 | 87 | 83 |

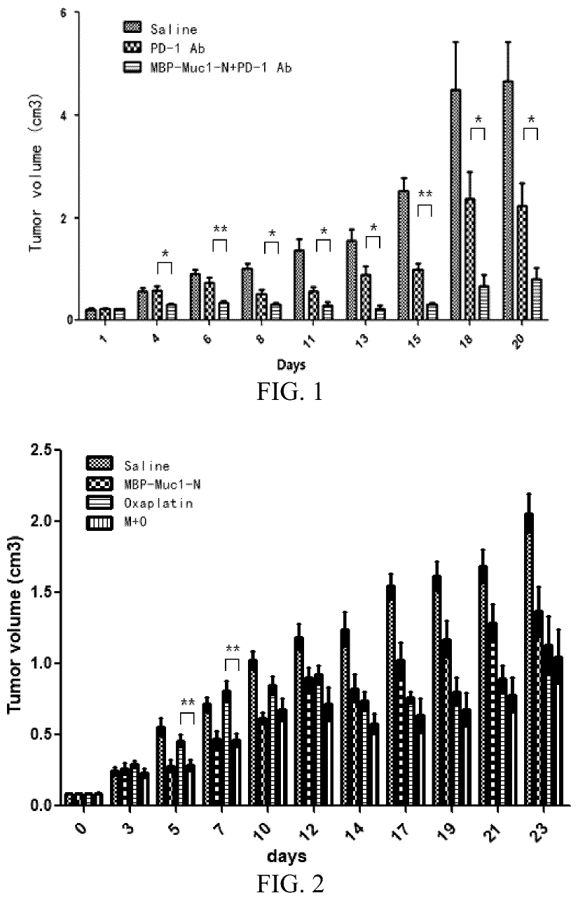

As can be seen from Table 1 and FIG. 1, the PD-1 antibody in combination with MBP-Muc1-N could significantly eliminate tumors compared to the use of the PD-1 antibody alone. The tumors were significantly reduced 3 days, 5 days, 7 days, 10 days, 12 days, 14 days, 17 days, and 19 days after the injection of the PD-1 antibody in combination with MBP-Muc1-N. The tumors were reduced from 0.58+0.21 cm³ to 0.31+0.06 cm³; from 0.74+0.24 cm³ to 0.34+0.99 cm³; from 0.52+0.20 cm³ to 0.30+0.10 cm³; from 0.57+0.19 cm³ to 0.30+0.15 cm³; from 0.89+0.42 cm³ to 0.22+0.15 cm³; from 0.98+0.31 cm³ to 0.30+0.11 cm³; from 2.34+1.30 cm³ to 0.66+0.56 cm³; and from 2.22+1.11 cm³ to 0.80+0.55 cm³, respectively. The p values were 0.024, 0.008, 0.049, 0.023, 0.01, 0.002, 0.022, and 0.025, respectively, with significant differences.

### 4. Conclusion

The recombinant MBP-MUC1-N fusion protein vaccine has a certain inhibitory effect on tumors. It can more effectively inhibit the growth of MC38 colon cancer tumors, and can improve the tumor cure rate, achieving the maximal cure rate, after being administered in combination with the immune checkpoint inhibitor PD-1 antibody.

### Example 2. Treatment of MC38 Colon Cancer in Combination with Chemotherapeutic Drug Oxaliplatin

### 1. Materials

Experimental reagents: the recombinant MBP-MUC1-N fusion protein prepared by using the method of the present application, and the vaccine was prepared by adopting a conventional vaccine preparation method; oxaliplatin was purchased from Sigma-Aldrich (USA); the normal saline for injection was purchased from Beijing Tiantan Biological Products Co., Ltd.

Experimental animals: C57 BL/6 mice were purchased from Beijing Huafukang Biotechnology Co., Ltd.

### 2. Methods

### 2.1 Immunization of Mice

Mice were weighed and randomly divided into groups of 8. MC38 colon cancer cells were diluted with an IMDM basal medium and inoculated in the right axilla of the C57BL/J mice according to 5×10⁵ cells/mouse, with a total amount of 100 μL/mouse, for modeling. After the tumor diameter reached 5 mm, the mice were divided into 4 groups, namely a normal saline control group (NS group), an oxaliplatin injection group (oxaliplatin group), a recombinant MBP-MUC1-N fusion protein vaccine group (vaccine group), and an oxaliplatin in combination with recombinant MBP-MUC1-N fusion protein vaccine formulation group (combination group). On day 3, day 7, day 10, day 14, and day 17 of tumor bearing, the recombinant MBP-MUC1-N fusion protein vaccine formulation was intramuscularly injected at a dose of 100 pg/mouse (diluted with NS, with a total amount of 200 μL) for immunization, and oxaliplatin was intraperitoneally injected at a dose of 3 mg/kg (diluted with 12% DMSO).

### 3. Results

TABLE 2
Tumor weights (g), tumor inhibition rates
(%) and tumor cure rates in mice (%)

| | Tumor | Tumor | Tumor |
|---|---|---|---|
| | weight | inhibition rate | cure rate |
| NS group | 2.42 ± 0.41 | 0 | 0 |
| Oxaliplatin group | 1.20 ± 0.39 | 50 | 12.5 |
| Vaccine group | 1.76 ± 0.53 | 27 | 0 |
| Combination group | 1.02 ± 0.48 | 58 | 37.5 |

As can be seen from Table 2 and FIG. 2, compared to the vaccine monotherapy group, oxaliplatin in combination with MBP-Muc1-N could significantly eliminate tumors compared to the use of oxaliplatin alone. The tumors were very significantly reduced 5 days and 7 days after the injection of oxaliplatin in combination with MBP-Muc1-N. The tumors were reduced from 0.46+0.12 cm³ to 0.29+0.12 cm³; and from 0.81+0.20 cm³ to 0.46+0.12 cm³.

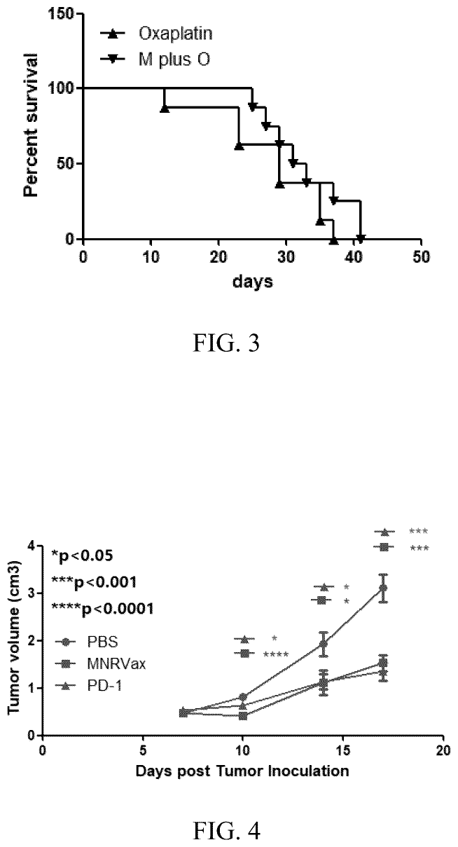

As shown in FIG. 3, oxaliplatin in combination with MBP-Muc1-N could prolong the life of mice bore cancer compared to the use of oxaliplatin alone. The survival time of the mice suffering from the cancer was prolonged from 29 days to 32 days.

### 4. Conclusion

After the recombinant MBP-MUC1-N fusion protein vaccine is administered in combination with oxaliplatin, the growth of MC38 colon cancer tumors can be more effectively inhibited, and the tumor cure rate can be improved, achieving the maximal cure rate.

The combined use of MBP-Muc1-N with oxaliplatin can significantly eliminate colon cancer tumors and prolong the life.

### Example 3. Vaccine Prepared by Three-Step Purification in Treatment of MC38 Colon Cancer Alone

### 1. Materials

Experimental reagents: a recombinant MBP-MUC1-N fusion protein expression strain was prepared using the method of the present application; the anti-PD-1 antibody was purchased from Bioxcell; MC38 colon cancer cells were purchased from National Experimental Cell Resource Center; the normal saline for injection was purchased from Beijing Tiantan Biological Products Co., Ltd. Experimental animals: C57 BL/6J mice were purchased from Beijing Huafukang Biotechnology Co., Ltd.

### 2. Methods

### 2.1 Production of MBP-MUC1-N Fusion Proteins

2.1.1 Fermentation formula: 5.0 g/L yeast extract, 10.0 g/L tryptone, 5.0 g/L NaCl, 3.5 g/L KH₂PO₄, 6.6 g/L K₂HPO₄·3H₂O, 3.5 g/L (NH₄)₂HPO₄, 1.0 g/L MgSO₄·7H₂O, and 1 mL antifoaming agent, with the addition of sterilized and filtered 0.1 g/L VB1 and 10.0 g/L glucose after sterilization.

2.1.2 Fermentation procedures: seeds were inoculated according to a ratio of 1:1000, and cultured at 37° C. and 200 rpm for 14 h; and then, the seeds were inoculated into 2 L of a fermentation medium according to a ratio of 1:10, and cultured at 37° C. at a stirring speed of 400-700 rpm. The dissolved oxygen before induction was 30%, which was associated with stirring. The pH was 7.0, which was associated with phosphoric acid and aqueous ammonia. Induction began when OD600 nm value reached 30. IPTG was added to a final concentration of 0.5 mM, and induction culture was performed at 28° C. for 4 h. When glucose was lower than 1.0 g/L, a feeding medium (200 g/L yeast extract and 200 g/L glucose) was added at a rate of 1%-2%. After induction, sampling was performed, and the sample was subjected to ultrasonication followed by SDS-PAGE analysis. The results showed that the induced proteins were located in the supernatants after disruption, and the unit yields of the bacterial sludges were each 69 g/L.

### 2.1.3 Bacterial Cell Clarification and Disruption

After the bacterial cells were disrupted, the mixture was subjected to depth filtration and 0.2-micron filtration (after the depth filtration, the mixture was filtered using a 0.2 m² and 0.2-micron filter), and the turbidity of the clarified sample was less than 15 NTU (nephelometric turbility unit).

The bacteria-disrupting solution was 20 mL, and bacteria disruption was ultrasonically performed in an ice bath. The mixture was subjected to high-speed-low-temperature centrifugation followed by sterile filtration. The resulting mixture was left to stand at 4° C. for 0 h, 3 h, 6 h, and 24 h. The results showed that the total amounts of the proteins reached 100%, 86%, 75%, and 56% of that at 0 h, respectively.

### 2.1.4 Purification of MBP-MUC1-N Fusion Proteins

The clarified liquid was first treated with an affinity chromatography column Dextrin Beads 6FF from Smart-Lifesciences in a volume of 20 mL which equilibrated with a binding buffer 1 (20 mM Tris-HCl, 0.2 M NaCl, 1 mM EDTA, pH 7.5), and 30 mL of the supernatant after ultrasonication was loaded; the sample was then washed with the binding buffer, and finally the target protein was eluted with an elution buffer (20 mM Tris-HCl, 0.2 M NaCl, 1 mM EDTA, 10 mM maltose, pH 7.4).

Then, primary purification was performed by using Bestarose Q HP. The loading buffer for Bestarose Q HP was 50 mM Tris-HCl (pH 8.0), and the elution buffer was 50 mM Tris-HCl and 0.5 M NaCl (pH 8.0). Then, the purification was performed by POROS XS; the binding buffer was 20 mM citric acid-sodium citrate (pH 4.6), and the elution buffer was 20 mM citric acid-sodium citrate and 0.5 M NaCl (pH 4.6).

### 2.2 Immunization of Mice

Mice were weighed and randomly divided into groups of 10. MC38 colon cancer cells were diluted with an IMDM basal medium and inoculated in the right axilla of the C57BL/J mice according to 3×10⁶ cells/mouse, with a total amount of 100 μL/mouse, for modeling. After the tumor diameter reached 5 mm, the mice were divided into 4 groups, namely a PBS control group (NS group), an immune checkpoint inhibitor anti-PD-1 antibody injection group (PD-1 group), and a recombinant MBP-MUC1-N fusion protein vaccine group (vaccine group). On day 3, day 7, day 10, and day 14 of tumor bearing, the recombinant MBP-MUC1-N fusion protein vaccine formulation was intramuscularly injected at a dose of 50 pg/mouse (with a total amount of 100 μL) for immunization, and the immune checkpoint inhibitor anti-PD-1 antibody was intraperitoneally injected at a dose of 200 pg/mouse (with a total amount of 100 μL/mouse).

2.3 Comparison of inhibitory effects of MBP-MUC1-N fusion protein vaccine and anti-PD-1 antibody on MC38 colon cancer at recombinant therapeutic endpoint (mean tumor value greater than 3 cm³) The tumor graft inhibition rate and the tumor inhibition rate of the subcutaneous colon cancer in each group were calculated. The formula is as follows: [tumor inhibition rate=(mean tumor weight in control group −mean tumor weight in experimental group)/mean tumor weight in control group×100%]. [tumor cure rate=(number of mice without tumor/6 mice)×100^{%}].

### 3. Results

TABLE 3
Tumor weights (g), tumor inhibition rates
(%) and tumor cure rates in mice

| | Tumor | Tumor | Tumor |
|---|---|---|---|
| | weight | inhibition rate | cure rate |
| PBS group | 3.10 ± 0.93 | 0 | 0 |
| Vaccine group | 1.53 ± 0.55 | 40 | 40 |
| PD-1 group | 1.35 ± 0.60 | 80 | 20 |

As can be seen from Table 3 and FIG. 4, the MBP-Muc1-N vaccine treatment group could significantly eliminate tumors compared to the control group. The tumors were significantly reduced 3 days, 7 days and 10 days after the injection of the MBP-Muc1-N vaccine. The tumors were reduced from 0.82+0.10 cm³ to 0.41+0.17 cm³; from 1.93+0.79 cm³ to 1.11+0.83 cm³; and from 3.10+0.93 cm³ to 1.53+0.55 cm³, respectively. The p values were 0.00001, 0.036, and 0.0004, respectively, with significant differences. In contrast, the tumors were significantly reduced 3 days, 7 days, and 10 days after administration of the positive control PD-I antibody. The tumors were reduced from 0.82+0.10 cm³ to 0.63+0.17 cm³; from 1.93+0.79 cm³ to 1.13+0.52 cm³; and from 3.10+0.93 cm³ to 1.35+0.60 cm³, respectively. The p values were 0.010, 0.017, and 0.0001, respectively, with significant differences. The above indicates that the MBP-Muc1-N vaccine takes effect quickly, and the PD-I takes effect slowly.

### 4. Conclusion

The recombinant MBP-MUC1-N fusion protein vaccine can have a relatively good inhibitory effect on tumors, the effect of which is close to that of the immune checkpoint inhibitor PD-I antibody, but the onset time is earlier.

### Example 4. Effects of Different Buffers on Vaccine Treatment of MC38 Colon Cancer

### 1. Materials

Experimental reagents: the recombinant MBP-MUC1-N fusion protein was prepared by using the method of the present application, and the buffer salts included 20 mM acetic acid-sodium acetate (pH 5.0) and 150 mM NaCl (Ac5.0 group for short); 20 mM acetic acid-sodium acetate (pH 7.3) and 150 mM NaCl (Ac7.3 group for short); and 20 mM Tris salt (pH 7.3) and 150 mM NaCl (Tris7.3 group for short). The vaccine was prepared by adopting a conventional vaccine preparation method; PBS was purchased from Tiantan Biological Products Co., Ltd.

Experimental animals: C57 BL/6 mice were purchased from Beijing Huafukang Biotechnology Co., Ltd.

### 2. Methods

### 2.1 Immunization of Mice

Mice were weighed and randomly divided into groups of 10. MC38 colon cancer cells were diluted with an IMDM basal medium and inoculated in the right axilla of the C57BL/J mice according to 3×10⁶ cells/mouse, with a total amount of 100 μL/mouse, for modeling. After the tumor diameter reached 5 mm, the mice were divided into 4 groups, namely a PBS control group (PBS group), an Ac5.0 group, an Ac7.3 group, and a Tris7.3 group. On day 3, day 7, day 10, day 14, and day 17 of tumor bearing, the recombinant MBP-MUC1-N fusion protein vaccine formulation was intramuscularly injected at a dose of 100 pg/mouse.

### 3. Results

TABLE 4
Tumor weights (g), tumor inhibition rates
(%) and tumor cure rates (%) in mice

| | Tumor | Tumor | Tumor |
|---|---|---|---|
| | weight | inhibition rate | cure rate |
| PBS group | 3.02 ± 0.80 | 0 | 0 |
| Ac5.0 group | 3.03 ± 0.54 | −0.3 | 0 |
| Ac7.3 group | 3.18 ± 0.86 | −5.30 | 0 |
| Tris7.3 group | 1.98 ± 0.83 | 34.4 | 10% |

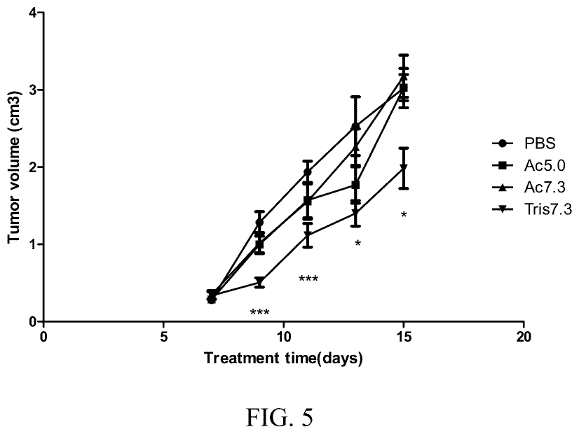

As can be seen from Table 4 and FIG. 5, compared to the PBS control group, the tumors in the Tris7.3 vaccine group were reduced from 3.02+0.80 cm³ to 1.98+0.83 cm³ (p=0.011); whereas the tumors in the Ac5.0 group and the Ac7.3 group were not reduced and were 3.03+0.54 cm³ and 3.18+0.86 cm³, respectively.

Compared to the PBS control group, the tumors were significantly reduced 2 days, 4 days, 6 days, and 8 days after the injection of the Tris7.3 vaccine. The tumors were reduced from 1.28+0.44 cm³ to 0.51+0.19 cm³, from 1.94+0.44 cm³ to 1.12+0.48 cm³, from 2.53+1.20 cm³ to 1.40+0.52 cm³, and from 3.02+0.80 cm³ to 1.98+0.83 cm³, respectively. The p values were 0.0002, 0.0009, 0.018, and 0.011, respectively, with significant differences.

The embodiments of the present disclosure have been described above. However, the present disclosure is not limited to the embodiments described above. Any modification, equivalent replacement, improvement, and the like made without departing from the spirit and principle of the present disclosure shall fall within the protection scope of the present disclosure.

1-17. (canceled)

18. A medicament for treating and/or preventing rectal cancer or colon cancer, wherein the medicament comprises a recombinant MBP-MUC1-N fusion protein vaccine and oxaliplatin, the fusion protein is set forth in SEQ ID NO. 3

19. The medicament according to claim 18, wherein the MBP-MUC1-N fusion protein vaccine comprising the fusion protein and a buffer system, and the buffer system comprises Tris salt and NaCl.

20. The medicament according to claim 19, wherein the buffer system comprises 20 mM Tris salt and 150 mM NaCl, with a pH of 7.3.

21. The medicament according to claim 18, wherein the medicament comprises a anti PD-1 antibody.

22. A method for preventing and/or treating rectal cancer or colon cancer, wherein the method comprises administering to a subject the medicament according to claim 18.

23. A method for preventing and/or treating rectal cancer or colon cancer, wherein the method comprises administering to a subject the medicament according to claim 19.

24. A method for preventing and/or treating rectal cancer or colon cancer, wherein the method comprises administering to a subject the medicament according to claim 20.

25. A method for preventing and/or treating rectal cancer or colon cancer, wherein the method comprises administering to a subject the medicament according to claim 21.

26. The method according to claim 22, which characterized in that the recombinant MBP-MUC1-N fusion protein vaccine is expressed by connecting maltose-binding protein MBP gene and human mucin MUC1-N gene in tandem, the nucleotide sequence of the MUC1-N gene is set forth in SEQ ID NO. 1, and the MBP gene is set forth in SEQ ID NO. 2.


SEQUENCE LISTING (3 sequences)

SEQ ID NO: 1 - DNA, 420 bp, unidentified
  source 1..420: mol_type = other DNA
ggtgttacttctgctcctgatactcgtcctgctcctggttctactgcaccgccagcacatggcgtgacgtctgcgccagatacccgtccggcaccgggttccaccgccccaccggcacacggcgtaacctccgcgccagacacccgtccagcgccaggttctaccgctccgcctgctcatggtgttacctctgccccggacactcgtccggctccaggttctactgccccgccagctcatggcgtcacttccgccccggatacccgtcctgccccgggctctactgcgcctccggctcacggcgttacctctgcaccggatactcgtccggctccgggctctaccgcaccacctgctcatggcgtaacgagcgctcctgatacccgtccggctccgggttccactgcacctccggcccac

SEQ ID NO: 2 - DNA, 1164 bp, unidentified
  source 1..1164: mol_type = other DNA
aaaatcgaagaaggcaaactggtgatctggatcaacggtgataagggttataacggtctggcggaagtaggcaagaaattcgaaaaagacaccggtatcaaagttaccgttgaacatccagacaaactggaagaaaaattccctcaggtggcggctaccggcgacggccctgatatcattttctgggcacatgatcgttttggcggttacgcgcagtctggcctgctggcagaaatcacgccggataaggcgttccaggacaaactgtacccttttacctgggacgcggtgcgttacaacggcaaactgatcgcttacccgatcgcagtggaagctctgtccctgatctacaataaggacctgctgccgaacccgcctaaaacgtgggaagaaatcccggccctggacaaagaactgaaagcaaaaggtaagagcgctctgatgttcaatctgcaggaaccgtacttcacttggccgctgatcgcagctgacggcggttatgcgtttaaatacgaaaacggtaaatatgacattaaggacgtcggcgttgataacgccggcgccaaagcgggcctgacctttctggtcgacctgatcaaaaacaaacacatgaacgctgacaccgattattctattgcggaggcggcttttaacaagggcgagaccgcaatgaccatcaacggtccgtgggcttggtctaacatcgacacctccaaagtaaattacggtgttaccgtcctgccgaccttcaaaggtcaaccgagcaaaccgttcgtgggcgtgctgtccgcaggtatcaacgctgcctccccaaacaaagagctggccaaagagttcctggaaaactatctgctgaccgacgaaggcctggaagctgttaataaagacaaaccgctgggtgctgttgcactgaaatcctatgaagaagaactggtcaaagatccgcgtattgccgccactatggagaacgcgcagaaaggtgaaatcatgccgaacatcccgcaaatgtccgctttttggtacgcggtgcgtaccgctgtaattaacgcggcgtccggtcgtcagactgtcgatgaagcgctgaaagatgctcagactaactctagctctaacaataacaataataacaacaacaacaatctgggtattgaaggtcgcatctct

SEQ ID NO: 3 - protein, 528 aa, synthetic construct
KIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELVKDPRIAATMENAQKGEIMPNIPQMSAFWYAVRTAVINAASGRQTVDEALKDAQTNSSSNNNNNNNNNNLGIEGRISGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAH